(19) United States
(12) Patent Application Publication
(10) Pub. No.: US 2022/0279842 A1
(43) Pub. Date: Sep. 8, 2022

(54) PORTABLE VAPORIZING APPARATUS AND A METHOD THEREOF

(71) Applicant: John Cleaon Moore, Camarillo, CA (US)

(72) Inventor: John Cleaon Moore, Camarillo, CA (US)

(21) Appl. No.: 17/688,630

(22) Filed: Mar. 7, 2022

Related U.S. Application Data
(60) Provisional application No. 63/157,523, filed on Mar. 5, 2021.

(51) Int. Cl. A24F 40/10; A24F 40/42; A61M 15/00

(52) U.S. Cl. CPC A24F 40/10; A24F 40/42; A61M 15/0085; A61M 15/0021

(57) ABSTRACT

A portable vaporizing apparatus for inhalation therapy and method of operation of the portable vaporizing apparatus are disclosed. The portable vaporizing apparatus includes a liquid cartridge comprising therapeutic substance, and ultrasonic activating tip removably coupled to proximal end of liquid cartridge. Furthermore, portable vaporizing apparatus includes mouthpiece having airflow outlet removably coupled to outer edge of proximal end of liquid cartridge. Thereafter, portable vaporizing apparatus includes power source removably coupled to distal end of liquid cartridge. The portable vaporizing apparatus receives ambient air via mouthpiece and power from power source to liquid cartridge. Further, portable vaporizing apparatus agitates ultrasonic waves in ultrasonic activating tip using received power.

Furthermore, portable vaporizing apparatus vaporizes therapeutic substance comprised in liquid cartridge, based on agitated ultrasonic waves and ambient air. Also, portable vaporizing apparatus creates aerosol from vaporized therapeutic substance, to pass into mouthpiece for breathing into airway of user.

### FIELD OF INVENTION

The embodiments of the present disclosure generally relate to a personal inhalation device. More particularly, the present disclosure relates to a portable vaporizing apparatus for inhalation therapy and a method of operation of the portable vaporizing apparatus.

### BACKGROUND OF THE INVENTION

The following description of related art is intended to provide background information pertaining to the field of the disclosure. This section may include certain aspects of the art that may be related to various features of the present disclosure. However, it should be appreciated that this section be used only to enhance the understanding of the reader with respect to the present disclosure, and not as admissions of prior art.

In general, various types of nebulizers may have been used in commerce for a variety of applications and are well known in the art. One common application for a nebulizer is in the medical industry. The nebulizer may be used in the medical industry for the effective delivery of medicines to a patient's lungs. The nebulizers can be used with spacers and holding chambers, and the nebulizers can also be fitted with adapters to provide positive expiratory pressure therapy, and/or positive airway pressure therapy.

Further, an aerosol may be a nebulous collection of atomized liquid droplets or fine powder particles, or vapor, often suspended in the air that can be available for inhalation. Aerosol particles can be solid or liquid fine particles and come in a variety of shapes. An aerosolized therapy may be an aerosolized liquid or powder using a nebulizer and humidifier, or the condensation aerosol that forms after vaporization of a substance, regardless of whether it is physiologically active. However, conventional apparatus such as nebulizer and humidifier machines may require connection to power and operation with a fixed place. The conventional apparatus may offer limited portability, though the conventional apparatus uses sonication energy to convert liquids to aerosols.

Therefore, there is a need in the art to provide a portable vaporizing apparatus for inhalation therapy and a method of operation of the portable vaporizing apparatus, for solving the shortcomings of the current apparatus.

### SUMMARY

This section is provided to introduce certain objects and aspects of the present invention in a simplified form that are further described below in the detailed description. This summary is not intended to identify the key features or the scope of the claimed subject matter.

In an aspect, the present disclosure discloses a portable vaporizing apparatus for inhalation therapy, the portable vaporizing apparatus includes a liquid cartridge comprising a therapeutic substance. The liquid cartridge includes a proximal end and a distal end. Further, the portable vaporizing apparatus includes an ultrasonic activating tip removably coupled to the proximal end of the liquid cartridge. Furthermore, the portable vaporizing apparatus includes a mouthpiece having an airflow outlet removably coupled to an outer edge of the proximal end of the liquid cartridge. Thereafter, the portable vaporizing apparatus includes a power source removably coupled to the distal end of the liquid cartridge. The portable vaporizing apparatus receives an ambient air via the mouthpiece and a power from the power source to the liquid cartridge. Further, the portable vaporizing apparatus agitates ultrasonic waves in the ultrasonic activating tip using the received power. Furthermore, the portable vaporizing apparatus vaporizes the therapeutic substance comprised in the liquid cartridge, based on the agitated ultrasonic waves and the ambient air. Also, the portable vaporizing apparatus creates an aerosol from the vaporized the therapeutic substance, to pass into the mouthpiece for breathing into an airway of a user.

In an embodiment, the therapeutic substance includes at least one of an agent, a medication, and a liquid.

In an embodiment, the ultrasonic activating tip comprises one or more piezoelectric transducers or atomizers configured to vaporize or atomize a substance with sonic or ultrasonic energy.

In an embodiment, the ultrasonic activating tip is a disposable consumable or as a rechargeable format replenished by the user with the therapeutic substance.

In another embodiment, the ultrasonic waves comprise a high-frequency which produces ultra-small diameter aerosols which are breathed into the airway to arrive at the microscopic alveoli of the user, where the highest possibility of direct transfer to the blood occurs.

In yet another embodiment, the aerosol comprises at least one of nicotine, a nicotine salt, a nicotine analog, a nicotine derivative, and a nicotine extract.

In another aspect, the present disclosure discloses a method for an operation of a portable vaporizing apparatus. The method includes receiving, by a portable vaporizing apparatus, an ambient air via a mouthpiece and a power from a power source to a liquid cartridge. Further, the method includes agitating, by a portable vaporizing apparatus, ultrasonic waves in an ultrasonic activating tip using the received power. Furthermore, the method includes vaporizing, by a portable vaporizing apparatus, the therapeutic substance comprised in the liquid cartridge, based on the agitated ultrasonic waves and the ambient air. Thereafter, the method includes creating by a portable vaporizing apparatus, an aerosol from the vaporized therapeutic substance, to pass into the mouthpiece for breathing into an airway of a user.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE ACCOMPANYING DRAWINGS

The accompanying drawings, which are incorporated herein, and constitute a part of this invention, illustrate exemplary embodiments of the disclosed methods and systems in which like reference numerals refer to the same parts throughout the different drawings. Components in the drawings are not necessarily to scale, emphasis instead being placed upon clearly illustrating the principles of the present invention. Some drawings may indicate the components using block diagrams and may not represent the internal circuitry of each component. It will be appreciated by those skilled in the art that the invention of such drawings includes the invention of electrical components, electronic components, or circuitry commonly used to implement such components.

FIG. 1 illustrates an exemplary perspective view of a portable vaporizing apparatus, in accordance with an embodiment of the present disclosure.

FIG. 2 illustrates an exemplary flow diagram of tobacco extraction, in accordance with an embodiment of the present disclosure.

FIG. 3 illustrates a flow chart depicting a method of operation of a portable vaporizing apparatus, in accordance with an embodiment of the present disclosure.

The foregoing shall be more apparent from the following more detailed description of the invention.

### DETAILED DESCRIPTION OF INVENTION

In the following description, for the purposes of explanation, various specific details are set forth in order to provide a thorough understanding of embodiments of the present disclosure. It will be apparent, however, that embodiments of the present disclosure may be practiced without these specific details. Several features described hereafter can each be used independently of one another or with any combination of other features. An individual feature may not address all of the problems discussed above or might address only some of the problems discussed above. Some of the problems discussed above might not be fully addressed by any of the features described herein.

The ensuing description provides exemplary embodiments only, and is not intended to limit the scope, applicability, or configuration of the disclosure. Rather, the ensuing description of the exemplary embodiments will provide those skilled in the art with an enabling description for implementing an exemplary embodiment. It should be understood that various changes may be made in the function and arrangement of elements without departing from the spirit and scope of the invention as set forth.

Specific details are given in the following description to provide a thorough understanding of the embodiments. However, it will be understood by one of ordinary skill in the art that the embodiments may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

Also, it is noted that individual embodiments may be described as a process which is depicted as a flowchart, a flow diagram, a data flow diagram, a structure diagram, or a block diagram. Although a flowchart may describe the operations as a sequential process, many of the operations can be performed in parallel or concurrently. In addition, the order of the operations may be re-arranged. A process is terminated when its operations are completed but could have additional steps not included in a figure. A process may correspond to a method, a function, a procedure, a subroutine, a subprogram, etc. When a process corresponds to a function, its termination can correspond to a return of the function to the calling function or the main function.

The word “exemplary” and/or “demonstrative” is used herein to mean serving as an example, instance, or illustration. For the avoidance of doubt, the subject matter disclosed herein is not limited by such examples. In addition, any aspect or design described herein as “exemplary” and/or “demonstrative” is not necessarily to be construed as preferred or advantageous over other aspects or designs, nor is it meant to preclude equivalent exemplary structures and techniques known to those of ordinary skill in the art. Furthermore, to the extent that the terms “includes,” “has,” “contains,” and other similar words are used in either the detailed description or the claims, such terms are intended to be inclusive—in a manner similar to the term “comprising” as an open transition word—without precluding any additional or other elements.

Reference throughout this specification to “one embodiment” or “an embodiment” or “an instance” or “one instance” means that a particular feature, structure, or characteristic described in connection with the embodiment is included in at least one embodiment of the present invention. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The terminology used herein is for the purpose of describing particular embodiments only and is not intended to be limiting of the invention. As used herein, the singular forms “a”, “an”, and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Embodiments of the present disclosure provide a portable vaporizing apparatus an inhalation therapy and method of operation of the portable vaporizing apparatus. The portable vaporizing apparatus may not include heating or combustion. The portable vaporizing apparatus may allow a greater application to administering drugs or other species that are temperature sensitive. The portable vaporizing apparatus may be used for the administration of allergy calming agents as an antihistamine and related in an emergency. The portable vaporizing apparatus may be used for tobacco inhalant technology. Since, the portable vaporizing apparatus operates at room temperature with no smoke generation, and hence no cancer-causing agents. Instead, this non-combusting device operation produces aerosols of extracted and condensed non-harmful tobacco leaf ingredients. The portable vaporizing apparatus may be a tobacco breathing device that uses ultrasonic aerosol in the form of the Sonicated Tobacco Product (STP).

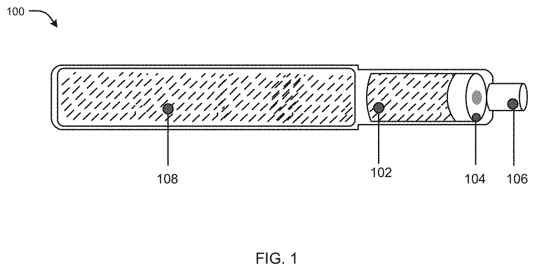

Referring to FIG. 1 illustrating an exemplary perspective view of a portable vaporizing apparatus 100, in accordance with an embodiment of the present disclosure, the portable vaporizing apparatus 100 may include a liquid cartridge 102, an ultrasonic activating tip 104, a mouthpiece 106, and a power source 108. The liquid cartridge 102 may include a therapeutic substance. Further, the liquid cartridge 102 may include a proximal end and a distal end. Furthermore, the ultrasonic activating tip 104 may be removably coupled to the proximal end of the liquid cartridge 102. The mouthpiece 106 may have an airflow outlet removably coupled to an outer edge of the proximal end of the liquid cartridge 102. The mouthpiece 106 may be detachable and replaceable. The mouthpiece 106 may include a cover to cover some or all of the mouthpiece 106. Further, the power source 108 may be removably coupled to the distal end of the liquid cartridge 102. The power source 108 may be removed from the liquid cartridge 102. The power may be provided by one or more batteries. The battery or batteries may be standard, replaceable batteries, or may be a rechargeable battery, and recharged with a recharging cord or similar means. The portable vaporizing apparatus 100 may include an outer main shell which may include one or more controls (not shown in FIG. 1), such as a button, slider, or switch, that may be used to turn the portable vaporizing apparatus 100 on or off, or control other functions. One or more lights, LEDs, or other indicators may be provided to indicate the status of the portable vaporizing apparatus 100, including, but not limited to, power status and operational status, and the like.

The therapeutic substance to be vaporized may be in the form of a liquid, gel, gas, solid, or the like. In several embodiments, the therapeutic substance includes, but are not limited to, one or more of a therapeutic substance, homeopathic or naturopathic formulations or remedies, serums, or the like. Particular substances may be chosen or selected for particular desired effects, therapies, or treatments, and substances have natural vaporization characteristics that are dependent on a variety of factors, including, but not limited to, temperature, air flow, substance composition, and chemical state. Selection of one or more substances for vaporization may be made based upon information obtained from other devices or systems, such as a breath analysis device that can capture and analyze the content of exhaled air, a health band, other detection devices, or information stored in or entered into in a health or wellness computer application, program or database on a computer or mobile device (e.g., smartphone, tablet computer), which can coordinate diagnosing this information and developing recommendations for the substance or substances to be used.

In an embodiment, the liquid cartridge 102 may include an internal, refillable chamber for holding the therapeutic substance to be vaporized. The refillable chamber may be filled by insertion of the substance through the opening with a removable cover, or other filling port. Vaporization may be achieved by means of one or more piezoelectric transducers or atomizers, providing sonic or ultrasonic vibration. Vapor may be emitted through the mouthpiece 106. In an instance, calibration and control of the atomization process (e.g., temperature, size of inlet openings, rate of vaporization, timer) may be controlled by the user manually using a control (not shown in FIG. 1) on the portable vaporizing apparatus 100, automatically controlled based upon a code or symbol (e.g., bar code) on the liquid cartridge 102, or remotely (such as by wireless connection to a computer or mobile device). liquid cartridge 102 itself may include the ultrasonic activating tip 104, and may be multiple use or single-use. The liquid cartridge 102 may be hermetically sealed, and made of any suitable material, including, but not limited to, glass, plastic, polymer, metal, or the like. The liquid cartridge 102 may be rigid, or flexible.

In an embodiment, the portable vaporizing apparatus 100 may receive ambient air via the mouthpiece 106 and power from the power source 108 to the liquid cartridge 102. The portable vaporizing apparatus 100 may agitate ultrasonic waves in the ultrasonic activating tip 104 using the received power. The ultrasonic waves may include a high-frequency which produces ultra-small diameter aerosols which are breathed into the airway to arrive at the microscopic alveoli of the user, where the highest possibility of direct transfer to the blood occurs. The ultrasonic activating tip 104 includes one or more piezoelectric transducers or atomizers configured to vaporize or atomize a substance with sonic or ultrasonic energy. The ultrasonic activating tip 104 may be a Microelectromechanical System (MEMS). The ultrasonic activating tip 104 may be a disposable consumable or as a rechargeable format replenished by the user with the therapeutic substance.

Further, the portable vaporizing apparatus 100 may vaporize the therapeutic substance in the liquid cartridge 102, based on the agitated ultrasonic waves and the ambient air. The therapeutic substance includes, but are not limited to, at least one of an agent, a medication, a liquid, and the like. Thereafter, the portable vaporizing apparatus 100 may create an aerosol from the vaporized therapeutic substance, to pass into the mouthpiece for breathing into an airway of a user. The aerosol may include at least one of, but are not limited to, nicotine, a nicotine salt, a nicotine analog, a nicotine derivative, a nicotine extract, and the like.

### Exemplary Scenario

One example may be an administration of allergy calming agents as an antihistamine and related in an emergency. The portable vaporizing apparatus 100 may work in conjunction with epi-pen technology.

Another example includes tobacco and inhalant technology. The portable vaporizing apparatus 100 may be presented as a sonicated tobacco product (STP). The portable vaporizing apparatus 100 may use a processed form of tobacco leaf, a modern extraction, and condensation practice, and merges it with an advanced delivery process based upon ultrasonic aerosol formation. The portable vaporizing apparatus 100 operate at room temperature, without combustion or heating. The process is based upon forming microscopic aerosols of the ingredients from tobacco leaves which are inhaled into the airway and deposited within the alveoli of the user's lungs. The tobacco leaf mixture may be contained within a compact device that is referred to as the liquid cartridge 102. The portable vaporizing apparatus 100 with the ultrasonic activating tip 104 which in total, may exist as a disposable consumable or as a rechargeable format replenished by the user with the chosen tobacco juice. The ultrasonic activating tip 104 is an ultrasonic device that may include a piezoelectric crystal. These materials electronically vibrate at frequencies from the very low to kilo and megahertz ranges. These vibrations interact with media in solids, liquids, and air/gases to create sound waves. The higher frequency vibration may produce aerosol droplets with measured diameters by micron scaling, usually, 50-100 um in low kilohertz (20-50 kHz) to finer as the frequency approaches megahertz. High-frequency sonication may be virtually undetected in sound by the human ear. The high frequency may produce ultra-small diameter aerosols which are breathed into the airway and ultimately arrive at the microscopic alveoli of the user where the highest possibility of direct transfer to the blood may occur.

In an instance, liquid sonicated processing may have been applied to foods to produce elixirs (blends). Using the sonication effects of microscopic waves moving through media to act in such liquids significantly reduces the time of processing and allows the user to capture internal ingredients bound within the chemistry and mechanical hindrances of the product's original form. In an example, consider plant cellulose, the cell wall may be one of the most robust representatives of difficult media to break down. Yet, plant cellulose breakdown may be critical to access the invaluable species within for energy and nutrients. While there may be more capacity to farm huge farms of fast-growing plants like bamboo, without the use of advanced technologies such as sonication, we are limited to effectively processing the large biomass that's produced. The cell walls of this fast-growing plant species have been used to create walls, bridges, and spears to separate and dominate human beings. The same barrier strength may present on a macro level may be observed microscopically. The sonication technique may use microscopic means to break through microscopic barriers.

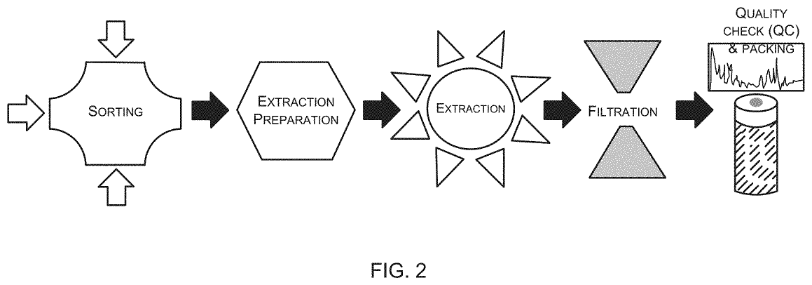

FIG. 2 illustrates an exemplary flow diagram of tobacco extraction, in accordance with an embodiment of the present disclosure. The portable vaporizing apparatus 100 may include, for example, tobacco vapor delivery. The tobacco juice may be derived directly from the leaf. The process of extraction and condensing desirable products may be stepwise, one that does not include high temperature or combustion. Sorting may involve the mixing of cultivars or varieties of tobacco, including location/farm. Further, the extraction preparation may include a mechanical breakdown of the incoming product that eases the process of extraction. Extraction may include capturing the desired constituents of the tobacco. Further, the filtration may include removing species that may harm the sonication process. Quality Check (QC) and packaging may include assaying target species and confirming the lack thereof of harmful.

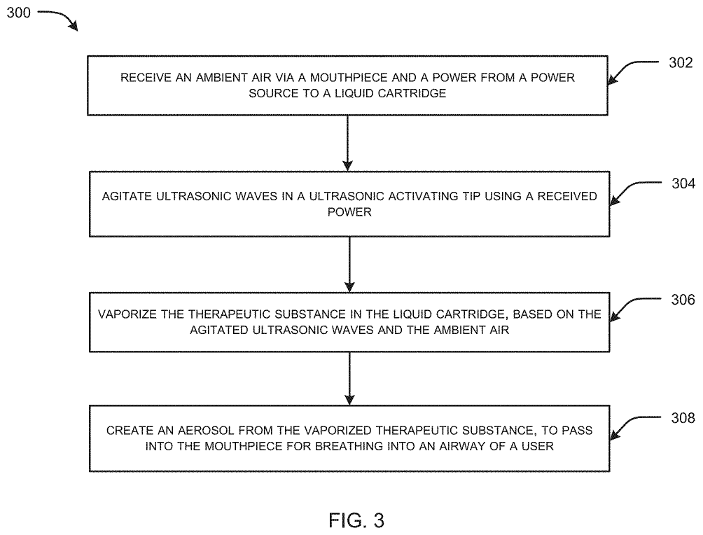

FIG. 3 illustrates a flow chart depicting method 300 of operation of a portable vaporizing apparatus, in accordance with an embodiment of the present disclosure.

At block 302, the method 300 may include receiving, by the portable vaporizing apparatus 100, an ambient air via the mouthpiece 106 and a power from a power source 108 to the liquid cartridge 102.

At block 304, the method 300 may include agitating, by the portable vaporizing apparatus 100, ultrasonic waves in an ultrasonic activating tip 104 using the received power.

At block 306, the method 300 may include vaporizing, by the portable vaporizing apparatus 100, the therapeutic substance comprised in the liquid cartridge 102, based on the agitated ultrasonic waves and the ambient air.

At block 308, the method 300 may include creating, by the portable vaporizing apparatus 100, an aerosol from the vaporized therapeutic substance, to pass into the mouthpiece 106 for breathing into an airway of a user.

Embodiments of the present disclosure provide a portable vaporizing apparatus an inhalation therapy and method of operation of the portable vaporizing apparatus. The portable vaporizing apparatus may not include heating or combustion. The portable vaporizing apparatus may allow a greater application to administering drugs or other species that are temperature sensitive. The portable vaporizing apparatus may be used for the administration of allergy calming agents as an antihistamine and related in an emergency. The portable vaporizing apparatus may be used for tobacco inhalant technology. Since, the portable vaporizing apparatus operates at room temperature with no smoke generation, and hence no cancer-causing agents. Instead, this non-combusting device operation produces aerosols of extracted and condensed non-harmful tobacco leaf ingredients. The portable vaporizing apparatus may be a tobacco breathing device that uses ultrasonic aerosol in the form of the Sonicated Tobacco Product (STP).

While considerable emphasis has been placed herein on the preferred embodiments, it will be appreciated that many embodiments can be made and that many changes can be made in the preferred embodiments without departing from the principles of the invention. These and other changes in the preferred embodiments of the invention will be apparent to those skilled in the art from the disclosure herein, whereby it is to be distinctly understood that the foregoing descriptive matter to be implemented merely as illustrative of the invention and not as a limitation.

What is claimed is:

1. A portable vaporizing apparatus for an inhalation therapy, the portable vaporizing apparatus comprising:
a liquid cartridge comprising a therapeutic substance, wherein the liquid cartridge comprises a proximal end and a distal end;
an ultrasonic activating tip removably coupled to the proximal end of the liquid cartridge;
a mouthpiece having an airflow outlet removably coupled to an outer edge of the proximal end of the liquid cartridge;
a power source removably coupled to the distal end of the liquid cartridge, wherein the portable vaporizing apparatus is configured to:
receive an ambient air via the mouthpiece and a power from the power source to the liquid cartridge;
agitate ultrasonic waves in the ultrasonic activating tip using the received power;
vaporize the therapeutic substance in the liquid cartridge, based on the agitated ultrasonic waves and the ambient air; and
create an aerosol from the vaporized therapeutic substance, to pass into the mouthpiece for breathing into an airway of a user.

2. The portable vaporizing apparatus as claimed in claim 1, wherein the therapeutic substance comprises at least one of an agent, a medication, and a liquid.

3. The portable vaporizing apparatus as claimed in claim 1, wherein the ultrasonic activating tip comprises one or more piezoelectric transducers or atomizers configured to vaporize or atomize a substance with sonic or ultrasonic energy.

4. The portable vaporizing apparatus as claimed in claim 1, wherein the ultrasonic activating tip is a disposable consumable or as a rechargeable format replenished by the user with the therapeutic substance.

5. The portable vaporizing apparatus as claimed in claim 1, wherein the ultrasonic waves comprise a high-frequency which produces ultra-small diameter aerosols which are breathed into the airway to arrive at the microscopic alveoli of the user, where the highest possibility of direct transfer to the blood occurs.

6. The portable vaporizing apparatus as claimed in claim 1, wherein the aerosol comprises at least one of nicotine, a nicotine salt, a nicotine analog, a nicotine derivative, and a nicotine extract.

7. A method of operation of a portable vaporizing apparatus, the method comprising:
receiving, by a portable vaporizing apparatus, an ambient air via a mouthpiece and a power from a power source to a liquid cartridge;
agitating, by the portable vaporizing apparatus, ultrasonic waves in an ultrasonic activating tip using the received power;
vaporizing, by the portable vaporizing apparatus, the therapeutic substance comprised in the liquid cartridge, based on the agitated ultrasonic waves and the ambient air; and
creating, by the portable vaporizing apparatus, an aerosol from the vaporized therapeutic substance, to pass into the mouthpiece for breathing into an airway of a user.

8. The method as claimed in claim 7, wherein the therapeutic substance comprises at least one of an agent, a medication, and a liquid.

9. The method as claimed in claim 7, wherein the ultrasonic activating tip comprises one or more piezoelectric transducers or atomizers configured to vaporize or atomize a substance with sonic or ultrasonic energy.

10. The method as claimed in claim 7, wherein the ultrasonic activating tip is a disposable consumable or as a rechargeable format replenished by the user with the therapeutic substance.

11. The method as claimed in claim 7, wherein the ultrasonic waves comprise a high-frequency which produces ultra-small diameter aerosols which are breathed into the airway to arrive at the microscopic alveoli of the user, where the highest possibility of direct transfer to the blood occurs.

12. The method as claimed in claim 7, wherein the aerosol comprises at least one of nicotine, a nicotine salt, a nicotine analog, a nicotine derivative, and a nicotine extract.
